(19) United States
(12) Patent Application Publication
(10) Pub. No.: US 2013/0280186 A1
(43) Pub. Date: Oct. 24, 2013

(54) Polymer Systems

(71) Applicants: Robert D'Ambrogio, Princeton, NJ (US); Rensl Dillon, Ewing, NJ (US); David B. Viscio, Prescott, AZ (US); James G. Masters, Ringoes, NJ (US)

(72) Inventors: Robert D'Ambrogio, Princeton, NJ (US); Rensl Dillon, Ewing, NJ (US); David B. Viscio, Prescott, AZ (US); James G. Masters, Ringoes, NJ (US)

(73) Assignee: Colgate-Palmolive Company, New York, NY (US)

(21) Appl. No.: 13/997,333

(22) PCT Filed: Dec. 23, 2010
(86) PCT No.: PCT/US10/61961

§ 371 Date: Jun. 24, 2013

(51) Int. Cl. A61K 8/73; A61Q 11/00; A61K 8/81

(52) U.S. Cl. CPC A61K 8/736; A61K 8/731; A61K 8/8147; A61Q 11/00
USPC 424/52; 424/49; 424/58

(57) ABSTRACT

Disclosed herein are polymer systems and compositions comprising polymer systems, and methods of preparing and using the same.

### BACKGROUND

Some individuals experience oral dryness on a frequent basis. Dry mouth is typically due to inadequate saliva production and effects many individuals. Dry mouth may be caused by several different factors including but not limited genetics, systemic illness, reactions to medications and aging. The degree to which an individual can experiences dry mouth can vary greatly. Stimulation of saliva production and use of oral moisturizers and substitute saliva are used to reduce dry mouth. Saliva production may be stimulated by various modalities such as mechanical, chemical, electrical and pharmacological.

Of the oral moisturizer products commercially available, the degree and duration of effectiveness varies. Thus, there exists a need for more effective, longer lasting oral rinses and mouthwashes that can be used to moisten the oral cavity and reduce the symptoms of dry mouth in individuals.

### SUMMARY

Some embodiments of the present invention provide a polymer system comprising: carboxymethyl cellulose; xanthan gum; carbomer; and a chitosan derivative.

Other embodiments provide compositions comprising any one of the polymer systems described herein and an orally acceptable carrier.

Further embodiments provide methods of treating or preventing a condition of the oral cavity comprising, contacting an oral cavity surface of a subject in need thereof, with any one of the compositions described herein.

### DETAILED DESCRIPTION

As used throughout, ranges are used as shorthand for describing each and every value that is within the range. Any value within the range can be selected as the terminus of the range.

In addition, all references cited herein are hereby incorporated by reference in their entireties.

In the event of a conflict in a definition in the present disclosure and that of a cited reference, the present disclosure controls.

In some embodiments, the present invention provides a polymer system comprising: one or more anionic mucoadhesive polysaccharide polymers; an acrylate polymer; and a chitosan derivative; wherein the weight ratio of anionic mucoadhesive polysaccharide polymer to acrylate polymer to chitosan derivative is about 2:1:6. In some embodiments, at least one of said one or more anionic mucoadhesive polysaccharide polymers is carboxymethyl cellulose. In some embodiments, at least one of said one or more anionic mucoadhesive polysaccharide polymers is xanthan gum. In some embodiments, the acrylate polymer is carbomer.

In some embodiments, the present invention provides a polymer system comprising: carboxymethyl cellulose; xanthan gum; carbomer; and a chitosan derivative. In some embodiments, the weight ratio of carboxymethyl cellulose to xanthan gum to carbomer to chitosan derivative is from about 1:1:1:2 to about 1:1:1:10. In some embodiments, the weight ratio of carboxymethyl cellulose to xanthan gum to carbomer to chitosan derivative is about 1:1:1:6. In some embodiments, the carboxymethyl cellulose, xanthan gum, carbomer and chitosan derivative are present in approximately equal parts, by weight.

In some embodiments, the present invention provides a toothpaste composition comprising: a polymer system comprising: one or more anionic mucoadhesive polysaccharide polymers in a total amount of from about 0.8 to about 2.4%, by weight of the composition; an acrylate polymer in the amount of from about 0.4 to 1.2%, by weight of the composition; a chitosan derivative in the amount of from about 1 to 10%, by weight of the composition; and an orally acceptable carrier. In some embodiments, the one or more anionic mucoadhesive polysaccharide polymers is present in a total amount of from about 1.2 to about 2%, by weight of the composition; an acrylate polymer is present in the amount of from about 0.6 to 1%, by weight of the composition; and a chitosan derivative is present in the amount of from about 3 to 7%, by weight of the composition.

In some embodiments, one or more anionic mucoadhesive polysaccharide polymers is present in the amount of about 1.6%, by weight of the composition; an acrylate polymer is present in the amount of about 0.8%, by weight of the composition; and a chitosan derivative is present in the amount of about 5%, by weight of the composition.

Some embodiments provide compositions wherein a first anionic mucoadhesive polysaccharide polymer is present in the amount of about 0.8%, by weight of the composition; and a second anionic mucoadhesive polysaccharide polymer is present in the amount of about 0.8%, by weight of the composition.

Some embodiments provide compositions comprising a polymer system as described herein; and an orally acceptable carrier. In some embodiments, the polymer system comprises from about 2 to about 14%, by weight, of the composition. In some embodiments, the polymer system comprises from about 5 to about 10%, by weight, of the composition. In some embodiments, the polymer system comprises from about 7 to about 8%, by weight, of the composition. In some embodiments, the polymer system comprises about 7.5%, by weight, of the composition. In some embodiments, the compositions are non-solid compositions.

As used herein, the term “non-solid composition” refers to a composition having the ability to take on the shape of its container.

In some embodiments, the present invention provides compositions comprising: from about 0.4 to 1.2%, by weight, carboxymethyl cellulose. In some embodiments, the composition comprises: from about 0.4 to 1.2%, by weight, xanthan gum. In some embodiments, the composition comprises: from about 0.4 to 1.2%, by weight, carbomer. In some embodiments, the composition comprises: from about 1 to 10%, by weight, of a chitosan derivative.

In some embodiments, the composition comprises: from about 0.4 to 1.2%, by weight, carboxymethyl cellulose; from about 0.4 to 1.2%, by weight, xanthan gum; from about 0.4 to 1.2%, by weight, carbomer; and from about 1 to 10%, by weight, of a chitosan derivative.

In some embodiments, the present invention provides compositions comprising: from about 0.5 to 1.1%, by weight, carboxymethyl cellulose. In some embodiments, the composition comprises: from about 0.5 to 1.1%, by weight, xanthan gum. In some embodiments, the composition comprises: from about 0.5 to 1.1%, by weight, carbomer. In some embodiments, the composition comprises: from about 2 to 8%, by weight, of a chitosan derivative.

In some embodiments, the composition comprises: from about 0.5 to 1.1%, by weight, carboxymethyl cellulose; from about 0.5 to 1.1%, by weight, xanthan gum; from about 0.5 to 1.1%, by weight, carbomer; and from about 2 to 8%, by weight, of a chitosan derivative.

In some embodiments, the present invention provides compositions comprising: from about 0.6 to 1%, by weight, carboxymethyl cellulose. In some embodiments, the composition comprises: from about 0.6 to 1%, by weight, xanthan gum. In some embodiments, the composition comprises: from about 0.6 to 1%, by weight, carbomer. In some embodiments, the composition comprises: from about 3 to 7%, by weight, of a chitosan derivative.

Yet further embodiments provide compositions comprising: from about 0.6 to 1%, by weight, carboxymethyl cellulose; from about 0.6 to 1%, by weight, xanthan gum; from about 0.6 to 1%, by weight, carbomer; and from about 3 to 7%, by weight, of a chitosan derivative. Still other embodiments provide compositions comprising: from about 0.7 to 0.9%, by weight, carboxymethyl cellulose; from about 0.7 to 0.9%, by weight, xanthan gum; from about 0.7 to 0.9%, by weight, carbomer; and from about 4 to 6%, by weight, of a chitosan derivative.

In some embodiments, the present invention provides compositions comprising about 0.8%, by weight, carboxymethyl cellulose. In some embodiments, the composition comprises about 0.8%, by weight, xanthan gum. In some embodiments, the composition comprises about 0.8%, by weight, carbomer. In some embodiments, the composition comprises about 5%, by weight, of a chitosan derivative.

In some embodiments, the composition comprises: about 0.8%, by weight, carboxymethyl cellulose; about 0.8%, by weight, xanthan gum; about 0.8%, by weight, carbomer; and about 5%, by weight, of a chitosan derivative. In some embodiments, the chitosan derivative is a cationic chitosan derivative. In some embodiments, the chitosan derivative is selected from: dicarboxylic acid chitosan; chitin-glucan; and carboxymethyl chitosan. In some embodiments, the chitosan derivative is carboxymethyl chitosan.

Some embodiments provide a method of treating or preventing a disease or condition of the oral cavity comprising contacting an oral cavity surface of a subject in need thereof, with any one of the polymer systems or compositions described herein.

In some embodiments, the compositions further comprise trimethylglycine. In some embodiments, the compositions further comprise a humectant. In some embodiments, the humectant is selected from: glycerin; sorbitol; and combinations thereof. In other embodiments, the humectant is present in an amount greater than about 40%, by weight. In some embodiments, the humectant is present in an amount greater than about 50%, by weight.

In some embodiments, the composition further comprises a plasticizer. In other embodiments, the plasticizer is selected from: polyethylene glycol; propylene glycol; xylitol; triacetin; triethyl citrate; mineral oil; castor oil; vegetable oil; and a combination of two or more thereof. In some embodiments, the composition further comprises one or more components selected from a fluoride ion source; a tartar control agent; a buffering agent; an abrasive; and a combination of two or more thereof.

In some embodiments, the composition is a dentifrice. In some embodiments, the dentifrice is selected from: a toothpaste and a mouthwash/mouthrinse. In some embodiments, the dentifrice is a toothpaste. In some embodiments, wherein the polymer system is incorporated in a mouthwash or mouthrinse, the concentration of each polymer may be less than that used in a toothpaste. In some embodiments, wherein the polymer system is incorporated in a mouthwash or mouthrinse, the concentration of each polymer may be reduced by a factor of 8 to 10.

Some embodiments provide a method of treating or preventing a condition of the oral cavity comprising, contacting an oral cavity surface of a subject in need thereof, with any one of the compositions described herein. In some embodiments, the condition of the oral cavity is xerostomia.

In some embodiments, the compositions comprise a blend of polymers which form the polymer system that are mucoadhesive and friction reducing in nature. In some embodiments, the polymer system has characteristics which make it particularly useful when used as a dentifrice. In some embodiments, the compositions exhibit enhanced spreadability on oral surfaces to provide adhesion, lubrication and slippery mouthfeel.

In some embodiments, the polymer system combines both rigid and flexible polymers consisting of anionic mucoadhesive polysaccharide polymers such as carboxymethyl cellulose and xanthan gum, along with additional anionic polymers such as polyacrylates and cationic based derivatives of chitosan polysaccharide. In some embodiments, the polymers spread and anchor on the soft oral surfaces during use of the compositions. In other embodiments, the polymer combination physically entangles with the outer glycoprotein (mucin) layers of the soft tissue for improved substantivity which forms a physically stronger surface layer which is harder to wash away under the dynamic conditions usually encountered in the oral environment.

In some embodiments, the polymers are provided in amounts suitable to form mucoadhesive polymer networks which adhere to surfaces within the oral cavity that lubricate to provide relief from oral dryness. In some embodiments, the polymers form an interpenetrating network.

In some embodiments, a mucoadhesive humectant such as trimethylglycine which is known to not only have unique water binding properties but act as a covalent anchor further improves the performance of the dentifrice in relieving oral dryness.

In some embodiments, cationic chitosan derivatives such as carboxymethyl chitosan not only function as a permeation enhancer, but also improve polymeric mucoadhesive attachment for the mucoadhesive anionic polymers by acting as a covalent anchor, preconditioning the oral surface and enhancing deposition.

Anionic synthetic high molecular weight polymers of acrylic acid known as carbomer include homopolymers of acrylic acid, crosslinked with an allyl ether pentaerythritol, allyl ether of sucrose or allyl ether of propylene. Carbomer has a USP classification of “carbomer homopolymer Type A”. Carbomers have the ability to absorb, retain water and swell to many times their original volume. Carbomer codes (910, 934, 940, 941, 971, 974 and 934P,) are an indication of molecular weight and the specific components of the polymer.

Cationic carboxymethyl chitosan polymers are readily available. In some embodiments, the cationic carboxymethyl chitosan may be 0.5-1.5% active. In some embodiments the cationic carboxymethyl chitosan is about 1.0% active.

The combination of polymers in the dentifrice imparts upon the product desirable friction reducing properties.

Optionally, trimethylglycine may be included in the composition. Timethylglycine, also known as, 1-carboxy-N,N,N-trimethylglycine, N-trimethylglycine and 1-carboxy-N,N,N-trimethylmethanamium hydroxide, is a zwitterionic material and an example of a mucoadhesive humectant. Trimethylglycine, which has unique water binding properties and can act as a covalent anchor to mucosal surfaces and other polymers, may be included in the combination. The presence of trimethylglycine enhances lubricity and provides a smooth pleasant mouthfeel. The inclusion of effective amounts of trimethylglycine is optional. In some embodiments, the amount of the trimethylglycine in the composition is from about 0.5 to 8%, by weight. Some embodiments include 1 to 5%, by weight, trimethylglycine. In some embodiments, the trimethylglycine is present in the amount of from about 2 to 4%, by weight. In some embodiments, the trimethylglycine is present in the amount of about 3%, by weight.

In some embodiments, the compositions described herein are a dentifrice. In some embodiments, the dentifrice is a semi-solid dentifrice. In other embodiments, the semi-solid dentifrice is aqueous. As recognized by one of skill in the art, dentifrices having a polymer system as described herein also comprise a dentifrice base that may optionally include other materials in various proportions. Examples of suitable carriers for oral care compositions are disclosed in U.S. Pat. Nos. 6,669,929 to Boyd et al., U.S. Pat. No. 6,379,654 to Gebreselassie et al., and U.S. Pat. No. 4,894,220 to Nabi et al.

In the preparation of the semi-solid/gel dentifrice base there is utilized an orally acceptable vehicle, including a water-phase with humectants. Humectants useful herein include polyhydric alcohols such as glycerin, sorbitol, xylitol or low molecular weight PEGs, alkylene glycol such as polyethylene glycol or propylene glycol. In various embodiments, humectants are operable to prevent hardening of paste or gel compositions upon exposure to air. In various embodiments humectants also function as sweeteners. One or more humectants may be present in a total amount of about 1% to about 50%, for example about 2% to about 45% or about 5% to about 35%. The total humectant concentration of the compositions described herein is typically from about 30 to about 80% by weight of the composition, or from about 50 to about 70% by weight of the composition.

The base dentifrice may also contain an inorganic or a natural or synthetic thickener or gelling agent. Thickeners or gelling agents include inorganic thickening silicas such as amorphous silicas available from Huber Corporation under the trade designation Zeodent 165, Irish moss, iota-carrageenan, polyvinylpyrrolidone, carboxyvinyl polymers, cellulosic polymers such as hydroxyethyl cellulose, carboxymethyl cellulose (carmellose) and salts thereof (e.g., carmellose sodium), natural gums such as karaya, gum arabic and tragacanth, colloidal magnesium aluminum silicate, colloidal silica and mixtures thereof. Optionally, one or more thickening agents are optionally present in a total amount of about 0.01% to about 15%, in some embodiments about 0.1% to about 10%, in some embodiments about 0.10 to about 5% by weight, in some embodiments about 0.2% to about 5% by weight and in some embodiments about 0.2 to about 1% by weight.

Dentifrices typically contain surface active agents, humectants, viscosity modifying agents and/or thickeners, abrasives, solvents, such as water, flavoring and sweetening agents. Other components may also be included. As recognized by one of skill in the art, the oral care compositions optionally include other materials in addition to those components previously described, including for example, emollients, moisturizers, mouth feel agents and the like.

In various embodiments, a semi-solid/gel composition is provided within a single component or phase. In other embodiments, the composition includes both a first and a second component that are separately maintained. Maintaining the components separately requires only that the components are maintained in such a way as to substantially prevent the interaction of one component of the composition with another component of the composition. Typically, a dual component oral care composition is employed where there are one or more incompatible ingredients included in the composition. For example, if the composition comprises two incompatible active ingredients, it is advantageous to maintain them separately. The separation of components can be accomplished through any means known or to be discovered in the art and includes chemical, physical, and mechanical means of separation of any combination of these. For example, the first and second incompatible components may be combined but certain components are separately maintained by wrapping or encapsulating one or both in a protective film, coating, capsule, micelle, etc.

Abrasives such as silica, calcined alumina, sodium bicarbonate, calcium carbonate, dicalcium phosphate and calcium pyrophosphate may be included in the base dentifrice compositions used in the practice of the present invention. Other abrasives may also be suitable for use in the compositions described herein. Visually clear dentifrice compositions may be obtained by using an abrasive such as collodial silica, e.g., those sold under the trade designation Zeodent 115 available from the Huber Corporation or alkali metal aluminosilicate complexes (that is, silica containing alumina combined in its matrix) which have refractive indices close to the refractive indices of gelling agent-liquid (including water and/or humectant) systems used in dentifrice compositions. The abrasive is generally present in the base dentifrice composition in weight concentrations of about 3% to about 50% by weight.

In some embodiments, the polymer system is contained and/or delivered through a film or film flakes. In some embodiments, dentifrices comprise colloidal particles. In some embodiments, colloidal particles are present in films or film flakes in the range of 40 to 50%, measured by dry weight of the film or film flake.

In addition to other components and additives described above, other additives and components may be optionally included in the base dentifrice. These additives described in more detail below may be incorporated into the base dentifrice. It is understood that while general attributes of each of the above categories of materials may differ; there may be some common attributes, and any given material may serve multiple purposes within two or more of such categories of materials.

To prepare the dentifrice base, water, humectants, e.g. glycerin, sorbitol polyethylene glycol are dispersed in a conventional mixer until the mixture becomes a homogeneous gel phase. Into the gel phase is added the abrasive. These ingredients are mixed until a homogeneous phase is obtained. Thereafter the thickener, any flavor and surfactants are added and the ingredients mixed at high speed until vacuum of about 20 to 100 mmHg.

As noted above, the dentifrice may be a mouthwash or oral rinse, which is substantially liquid in character. The vehicle for mouthwashes and oral rinses is typically water, i.e. they are generally aqueous solutions. The total amount of water is typically in the range of from about 70 to about 99.9% by weight. Typically, at least 80% of the dentifrice is made up of a one or more of water, sorbitol, glycerine and propylene glycol. Some embodiments of the dentifrice include at least 90% being made up of one or more of water, sorbitol, glycerine and propylene glycol. Water typically makes up the largest portion of the dentifrice. Water may be present in an amount from about 20 to 90%, by weight. Some embodiments include about 60 to about 80% water, by weight. Other embodiments include 70 to 75% water, by weight. Yet other embodiments are about 70%, about 71%, about 72%, about 73%, about 74% or about 75% water, by weight.

Other humectants such as polyol and sugar alcohol solutions may be present in amount of from about 1 to 25% each by weight. In some embodiments, the humectant is sorbitol. Some embodiments have sorbitol present in the amount of from about 5 to about 20%, by weight. Some embodiments have sorbitol present in the amount of about 10 to about 15%, by weight. In some embodiments, sorbitol is present in the amount of about 10%, by weight. Reference to sorbitol herein refers to the material typically available commercially in 70% aqueous solutions.

In some embodiments, the present invention provides methods comprising rinsing the oral cavity with a composition as described herein. In some embodiments, the shear thinning properties of the composition increase the flow and thus area covered when agitated within the oral cavity. In some embodiments, a polymer film forms on the surface of the oral cavity following discharge of the composition.

In some embodiments, or more surface active agents or surfactants are incorporated into the compositions described herein, and may function as a surfactant, emulsifier, and/or foam modulator. In various embodiments, surface active agents achieve increased prophylactic action by thoroughly dispersing the active ingredient agents throughout the composition base. Surfactants may be used, for example, to help in cleaning the oral cavity surfaces through detergency, and to provide foam upon agitation, e.g., during brushing or gargling with a dentifrice composition, to achieve increased prophylactic action. Further, in various embodiments, surface active ingredients can improve the cosmetic appearance of the dentifrice base. Suitable surface active and emulsifying agents are preferably those that are reasonably stable throughout a wide pH range, including anionic, nonionic, zwitterionic and amphoteric organic synthetic detergents. In certain embodiments, one or more surfactants are present in the composition in the range of about 0.001% to about 5%, by weight; about 0.5% to about 4%, by weight; or from about 1% to about 3%, by weight.

Suitable anionic surfactants include water-soluble salts of C₈-₂₀ alkyl sulfates, sulfonated monoglycerides of C₈-20 fatty acids such as the sodium salt of the monsulfated monoglyceride of hydrogenated coconut oil fatty acids, sarcosinates, taurates and mixtures thereof. Illustrative examples of these and other surfactants are higher alkyl sulfates such as sodium lauryl sulfate, sodium coconut monoglyceride sulfonate, sodium lauryl sarcosinate, sodium lauryl isoethionate, sodium laureth carboxylate, alkyl aryl sulfonates such as sodium dodecyl benzenesulfonate, higher alkyl sulfoacetates, sodium lauryl sulfoacetate, higher fatty acid esters of 1,2-dihydroxy propane sulfonate, and the substantially saturated higher aliphatic acyl amides of lower aliphatic amino carboxylic acid compounds, such as those having 12 to 16 carbons in the fatty acid, alkyl or acyl radicals, amides are N-lauroyl sarcosine, and the sodium, potassium, and ethanolamine salts of N-lauroyl, N-myristoyl, or N-palmitoyl sarcosine, and the like and mixtures thereof.

Suitable nonionic surfactants include poloxamers, polyoxyethylene sorbitan esters, fatty alcohol ethoxylates, alkylphenol ethoxylates, tertiary amine oxides, tertiary phosphine oxides, dialkyl sulf oxides and mixtures thereof.

Suitable amphoteric surfactants include derivatives of C₈-₂₀ aliphatic secondary and tertiary amines having an anionic group such as carboxylate, sulfate, sulfonate, phosphate or phosphonate. A suitable example is cocoamidopropyl betaine.

Viscosity modifiers among those useful herein include mineral oil, petrolatum, clays and organomodified clays, silica and mixtures thereof. In various embodiments, such viscosity modifiers are operable to inhibit settling or separation of ingredients or to promote redispersibility upon agitation of a liquid composition.

Diluents among those useful herein include materials or combinations of materials that are operable to solubilize and/or suspend other components of the composition. In various embodiments, diluents are operable to adjust the viscosity of the composition, optionally in conjunction with viscosity modifiers (as discussed herein) and other components of the composition. In some embodiments, the composition is non-aqueous, i.e., does not contain appreciable amounts of chemically-unbound water.

In some embodiments, the composition comprises less than about 20%, less than about 15%, less than about 10%, or less than about 5%, by weight, water.

Some embodiments provide compositions comprising a pH modifying agent. Among those useful herein include acidifying agents to lower pH, basifying agents to raise pH, and buffering agents to control pH within a desired range. For example, one or more compounds selected from acidifying, basifying and buffering agents can be included to provide a pH of about 2 to about 10, or in various embodiments from about 2 to about 8, from about 3 to about 9, from about 4 to about 8, from about 5 to about 7, from about 6 to about 10, and from about 7 to about 9. Any orally acceptable pH modifying agent can be used, including without limitation carboxylic, phosphoric and sulfonic acids, acid salts (e.g., monosodium citrate, disodium citrate, monosodium malate, etc.), alkali metal hydroxides such as sodium hydroxide, carbonates such as sodium carbonate, bicarbonates, sesquicarbonates, borates, silicates, phosphates (e.g., monosodium phosphate, trisodium phosphate, pyrophosphate salts, etc.), imidazole and mixtures thereof. One or more pH modifying agents are optionally present in a total amount effective to maintain the composition in an orally acceptable pH range.

Mouth-feel agents include materials which impart a desirable texture or other feeling during use of the composition. Such agents include bicarbonate salts, which in various embodiments impart a “clean feel” to teeth and gums due to effervescence and release of carbon dioxide. Any orally acceptable bicarbonate can be used, including without limitation alkali metal bicarbonates such as sodium and potassium bicarbonates, ammonium bicarbonate, and mixtures thereof. One or more bicarbonate salts are optionally present in a total amount of 0.1% to about 50%, for example about 1% to about 20%.

In various embodiments, the dentifrice base comprises a formulation colorant that imparts a color to the composition. The colorants include both pigments and dyes. Useful formulation colorants include non-toxic water soluble dyes or pigment, such as, for example, metallic oxide “lakes.” Useful pigments include non-toxic, water insoluble inorganic pigments such as titanium dioxide, titanium dioxide coated mica (Timiron), chromium oxide greens, ultramarine blues and pinks, phthalocyanine green, and ferric oxides as well as water insoluble dye lakes prepared by extending calcium or aluminum salts of FD&C dyes on alumina such as FD&C Green #1 lake, FD&C Blue #2 lake, FD&C R&D #30 lake and FD&C # Yellow 15 lake. The pigments may have a flake size in the range of 5 to 1000 microns, and in some, such a those sole under trade designation Spectra bead, which are high molecular weight polyethylene embodiments, the range may be 250 to 500 microns. Classes of dyes which may be used are available from Micropowders powders permanently colored with dyes such as FD&C Blue #1 aluminum lake. In various embodiments, a colorant is provided at a level of about 0.5% to about 20% by weight, or about 1% to about 15% by weight, or about 3% to about 12% by weight.

In certain embodiments, a sweetening material is used. Sweeteners may be either or both natural and artificial sweeteners. Suitable sweetener include water soluble sweetening agents such as monosaccharides, disaccharides and polysaccharides such as xylose, ribose, glucose (dextrose), mannose, galactose, fructose (levulose), sucrose (sugar), maltose, water soluble artificial sweeteners such as the soluble saccharin salts, i.e., sodium saccharin, calcium saccharin, cyclamate salts such as sodium cyclamate, dipeptide based sweeteners, such a L-aspartic acid derived sweeteners, such as L-aspartyl-L-phenylalaine methyl ester (aspartame), xylitol, perillartien, D-tryptophan, sucralose, dihydrochalcones and the like. In general, the effective amount of sweetener is utilized to provide the level of sweetness desired for a particular composition, will vary with the sweetener selected. This amount will normally be about 0.001% to about 5% by weight of the composition. In some embodiments, the amount will be about 0.01% to about 2% by weight of the composition, preferably in concentrations of 0.01 to about 1%. In some embodiments, the sweetening material is sodium saccharin. In some embodiments, the sweetener is sodium saccharin and present in the amount of from about 0.01% to 0.5% by weight of the composition.

Flavor agents are known, such as natural and artificial flavors. These flavorings may be chosen from synthetic flavor oils and flavoring aromatics, and/or oils, oleo resins and extracts derived from plants, leaves, flowers, fruits and so forth, and combinations thereof. Menthol is the preferred flavorant. Representative flavor oils include: spearmint oil, cinnamon oil, peppermint oil, clove oil, bay oil, thyme oil, cedar leaf oil, oil of nutmeg, oil of sage, and oil of bitter almonds. These flavor agents can be used individually or in admixture. Commonly used flavors include mints such as peppermint, artificial vanilla, cinnamon derivatives, and various fruit flavors, whether employed individually or in admixture. Generally, any flavoring or food additive, such as those described in Chemicals Used in Food Processing, publication 1274 by the National Academy of Sciences, pages 63-258, may be used. Typically, flavorants if included are present at 0.01 to 3%, by weight. In some embodiments, flavoring may be present in about 0.2%, by weight. In some embodiments, flavoring may be present in the amount of about 1.2%, by weight.

Optional breath freshening agents may be provided. Any orally acceptable breath freshening agent can be used, including without limitation zinc salts such as zinc gluconate, zinc citrate and zinc chlorite, a-ionone and mixtures thereof. One or more breath freshening agents are optionally present in a breath freshening effective total amount.

In some embodiments the compositions include a fluoride ion source. In some embodiments, the fluoride ion source is selected from: potassium, sodium and ammonium fluorides and monofluorophosphates, stannous fluoride, indium fluoride and mixtures thereof.

Optionally, a stannous ion source may be included, for example, as a periodontal active, tartar control agent, anticaries agent or tooth desensitizer. Any orally acceptable stannous ion source can be used, including stannous fluoride, other stannous halides such as stannous chloride dihydrate, organic stannous carboxylate salts such as stannous formate, acetate, gluconate, lactate, tartrate, oxalate, malonate and citrate, stannous ethylene glyoxide and the like.

Optionally, the dentifrice base may include a tartar control (anticalculus) agent. Tartar control agents among those useful herein include phosphates and polyphosphates (for example pyrophosphates), polyaminopropanesulfonic acid (AMPS), polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-diphosphonic acid, ethane-1-hydroxy-1,1-diphosphonic acid (EHDP) and ethane-1-amino-1,1-diphosphonate, phosphonoalkane carboxylic acids and salts of any of these agents, for example their alkali metal and ammonium salts. Useful inorganic phosphate and polyphosphate salts include mono, dibasic and tribasic sodium phosphates, sodium tripolyphosphate, tetrapolyphosphate, mono-, di-, tri- and tetrasodium pyrophosphates, sodium trimetaphosphate, sodium hexametaphosphate and mixtures thereof, wherein sodium can optionally be replaced by potassium or ammonium. Other useful anticalculus agents include polycarboxylate polymers and polyvinyl methyl ether/maleic anhydride (PVME/MA) copolymers, such as those available under the tradename Gantrez from ISP, Wayne, N.J. In some embodiments, the tartar control agent may be present in an amount from about 0.01 to about 10%, by weight. In some embodiments, the tartar control agent is present in the amount of about 0.1 to 2%, by weight. In some embodiments, the tartar control agent is present in the amount of about 0.5 to 1%, by weight. In some embodiments, the tartar control agent is present in the amount of about 0.5%, by weight. In some embodiments, the tartar control agent is present in the amount of 1%, by weight.

Other optional additives include antimicrobial (e.g., antibacterial) agents. Any orally acceptable antimicrobial agent can be used, including Triclosan (5-chloro-2-(2,4-dichlorophenoxy)phenol); 8-hydroxyquinoline and salts thereof, zinc and stannous ion sources such as zinc citrate, zinc sulphate, zinc glycinate, sodium zinc citrate and stannous pyrophosphate; copper (II) compounds such as copper (II) chloride, fluoride, sulfate and hydroxide; phthalic acid and salts thereof such as magnesium monopotassium phthalate; sanguinarine; quaternary ammonium compounds, such as alkylpyridinium chlorides (e.g., cetylpyridinium chloride (CPC), and combinations of two or more thereof.

Also optional, saliva stimulating agent, useful for example in amelioration of dry mouth may be included. Any orally acceptable saliva stimulating agent can be used, including without limitation food acids such as citric, lactic, malic, succinic, ascorbic, adipic, fumaric, and tartaric acids, and mixtures thereof. One or more saliva stimulating agents are optionally present in a saliva stimulating effective total amount.

Some embodiments comprise a desensitizing agent. Optional desensitizing agents include, but are not limited to, potassium citrate, potassium chloride, potassium tartrate, potassium bicarbonate, potassium oxalate, potassium nitrate, strontium salts, and mixtures thereof.

Embodiments of the present invention are further described in the following examples. The examples are merely illustrative and do not in any way limit the scope of the invention as described and claimed.

### EXAMPLES

### Example 1

Table 1 (below) describes the formulation of Composition A, which is an exemplary composition of the present invention containing an exemplary polymer system of the present invention.

TABLE 1

| Ingredient | % w/w |
|---|---|
| Glycerin | 40 |
| Sorbitol | 16 |
| Silica | 6.3 |
| Carboxymethyl chitosan (1% | 5 |
| Active) | |
| PEG600 | 3 |
| Trimethylglycine | 3 |
| PEG 40 Castor Oil | 3 |
| Dentifrice Flavor | 1.2 |
| Sodium Carboxymethyl Cellulose | 0.8 |
| Xanthan Gum | 0.8 |
| Carbopol 974 | 0.8 |
| Sodium Monofluorophosphate | 0.8 |
| Tetrasodium Pyrophosphate | 0.5 |
| Sodium Saccharin | 0.5 |
| FD&C Blue No. 1 (1.25% Solution) | 0.1 |
| Dibasic Potassium Phosphate | 0.08 |
| Magnesium Chloride Hexahydrate | 0.06 |
| Potassium Dihydrogen Phosphate | 0.04 |
| Calcium Chloride | 0.02 |
| Water | q.s. |

### Example 2

This Example provides the ingredient listing for Comparative Example 1. Comparative Example 1 contains the following ingredients: polyethylene glycol, hydrated silica, water, sorbitol, flavor, sodium lauryl sulfate, sodium CMC, tetrasodium pyrophosphate, sodium saccharin, sodium fluoride and FD&C Blue no. 1.

### Example 3

This Example provides the ingredient listing for Comparative Example 2. Comparative Example 2 contains the following ingredients: glycerin, hydrated silica, water, sorbitol, flavor, thione complex, calcium disodium EDTA, propylparaben, methylparaben, xanthan gum, citric acid, dibasic sodium phosphate, xylitol, sodium monofluorphosphate and titanium dioxide.

### Example 4

Static and dynamic in vitro tests were developed to measure the degree of polymer adhesion to model mucosal surfaces and the propensity of polymers to reduce frictional effects on mucosal surfaces. Changes in coefficient of friction (COF) from baseline (artificial saliva treatment only) were compared after treating a model mucosal substrate with 2:1 slurries of artificial saliva: toothpaste followed by rinsing with deionized water.

The data described in Table 2 (below) demonstrates that Composition A forms an interpenetrating elastic network that adheres to oral surfaces. This is exemplified by a negative change in μk versus baseline.

The data also suggests that the polymer film produced by Composition A produces a very slippery surface above minimal perturbations in Normal force. This is exemplified by a large positive change in μk versus baseline.

The data described in Table 2 demonstrates that Composition A provides superior adhesion and slipperiness versus Comparative Example 1. Comparative Example 1, which contains a carboxymethyl cellulose polymer system, but one different from the polymer systems of the present invention, does not produce a change in static coefficient of friction (no deposition on mucosa) and little change in dynamic coefficient of friction (only slightly slippery surface).

TABLE 2

| | μs (% Change from | μk (% Change from |
|---|---|---|
| Composition | Baseline) | Baseline) |
| A | −29% | +52% |
| Comparative Example 1 | 0% | +11% |

Static friction coefficient (μs) = Neg. value indicates “sticky” deposition
Kinetic friction coefficient (μk) = Pos. value indicates “slippery” surface with moderate perturbation

### Example 5

A dynamic vapor sorption (DVS) method was used to measure moisture retention and, indirectly, polymer/ humectant retention on a model mucosa surface. This method involved treating a substrate with 2:1 artificial saliva: toothpaste solution for one minute followed by a water rinse. Changes in the mass of the substrate were then measured with changes in humidity from 0% to 90%. Table 3 (below) describes the results of this experiment. The results highlight significant differences between mucosa substrate treated with Composition A versus untreated mucosal substrate and also versus mucosa substrates treated with dentifrice formulations (Comparative Examples 1 and 2) that do not contain the polymer systems of the present invention. The data demonstrates that Composition A displays a greater capacity to hydrate oral soft tissue versus both Comparative Examples 1 and 2.

TABLE 3

| Sample | % Change in Mass |
|---|---|
| Untreated Model Mucosa | 13.6 |
| Composition A | 52.1 |
| Comparative Example 1 | 34.2 |
| Comparative Example 2 | 25.3 |

The static and dynamic in vitro results and DVS data described in Tables 2 and 3, correlate with slippery, sticky, and moist mouthfeel attributes from the respective test products. This data shows that compositions of the present invention provide greater mouthfeel, deposition of polymer on oral cavity surfaces and moisture retention, than Comparative Examples 1 and 2.

As those skilled in the art will appreciate, numerous changes and modifications may be made to the embodiments described herein without departing from the spirit of the invention. It is intended that all such variations fall within the scope of the appended claims.

What is claimed is:

1. A polymer system comprising:
one or more anionic mucoadhesive polysaccharide polymers;
an acrylate polymer; and
a chitosan derivative;
wherein the weight ratio of anionic mucoadhesive polysaccharide polymer to acrylate polymer to chitosan derivative is about 2:1:6.

2. The polymer system of claim 1, wherein at least one of said one or more anionic mucoadhesive polysaccharide polymers is carboxymethyl cellulose.

3. The polymer system of claim 1, wherein at least one of said one or more anionic mucoadhesive polysaccharide polymers is xanthan gum.

4. The polymer system of claim 1, wherein said acrylate polymer is carbomer and said chitosan derivative is selected from: dicarboxylic acid chitosan; chitin-glucan; and carboxymethyl chitosan.

5. (canceled)

6. The polymer system of claim 4, wherein said chitosan derivative is carboxymethyl chitosan.

7. (canceled)

8. A toothpaste composition comprising:
a polymer system comprising:
one or more anionic mucoadhesive polysaccharide polymers in a total amount of from about 0.8 to about 2.4%, by weight of the composition;
an acrylate polymer in the amount of from about 0.4 to 1.2%, by weight of the composition;
a chitosan derivative in the amount of from about 1 to 10%, by weight of the composition; and
an orally acceptable carrier.

9. The composition of claim 8, wherein the polymer system comprises:
one or more anionic mucoadhesive polysaccharide polymers in a total amount of from about 1.2 to about 2%, by weight of the composition;
an acrylate polymer in the amount of from about 0.6 to 1%, by weight of the composition; and
a chitosan derivative in the amount of from about 3 to 7%, by weight of the composition.

10. (canceled)

11. The composition of claim 8, wherein at least one of said one or more anionic mucoadhesive polysaccharide polymers is carboxymethyl cellulose and said one or more anionic mucoadhesive polysaccharide polymers is xanthan gum.

12. (canceled)

13. The composition of claim 11, wherein said chitosan derivative is carboxymethyl chitosan.

14. (canceled)

15. The composition of claim 8, further comprising trimethylglycine.

16. The composition of claim 8, further comprising a humectant.

17. The composition of claim 16, wherein the humectant is selected from: glycerin; sorbitol; and combinations thereof,

18. The composition of claim 16, wherein the humectant is present in an amount greater than about 40%, by weight.

19. The composition of claim 8, further comprising a plasticizer.

20. The composition of claim 19, wherein the plasticizer is selected from: polyethylene glycol; propylene glycol; xylitol; triacetin; triethyl citrate; mineral oil; castor oil; vegetable oil; and a combination of two or more thereof.

21. The composition of claim 8, further comprising one or more components selected from a fluoride ion source; a tartar control agent; a buffering agent; an abrasive; and a combination of two or more thereof.

22. The composition of claim 21, wherein at least one of the one or more components is a fluoride ion source selected from: stannous fluoride, sodium fluoride, potassium fluoride, sodium monofluorophosphate, sodium fluorosilicate, ammonium fluorosilicate, amine fluoride, ammonium fluoride, and a. combination of two or more thereof.

23. A method of treating or preventing a condition of the oral cavity comprising, contacting an oral cavity surface of a subject in need thereof, with a composition according to claim 8.

24. The method of claim 23, wherein said condition of the oral cavity is xerostomia.
